(19) United States
(12) Patent Application Publication
(10) Pub. No.: US 2005/0197538 A1
(43) Pub. Date: Sep. 8, 2005

(54) Reagent and sample introduction plunger device for a syringe

(75) Inventors: John R. Leaton, Houston, TX (US); William F. Danner, Baton Rouge, TX (US)

Correspondence Address: KEELING HUDSON LLC, P.O. BOX 70103, HOUSTON, TX, 77270, US

(73) Assignee: Valco Instruments Company, Inc.

(21) Appl. No.: 11/065,877

(22) Filed: Feb. 25, 2005

Related U.S. Application Data
(60) Provisional application No. 60/550,551, filed on Mar. 5, 2004.

(51) Int. Cl. A61B 19/00

(52) U.S. Cl. 600/218

(57) ABSTRACT

A plunger has an orifice therethrough with a fixed given or variable orifice. The plunger is mounted to a commercially available syringe that expands the functionality of the syringe and its applications. The plunger allows for reagents and flushing of cleaning fluids to be added to the syringe without having to move the plunger up and down from vessel to vessel. The plunger also allows the use of a multiplex valving assembly to be added for air incorporation into the sample or liquid path. This is for use with sample array work and for cleaning gas or liquid samples from the syringe. Samples can be introduced from the closed part of the end of the syringe through the top of the plunger assembly. Many chemical and bio-related applications can be performed where it was not possible to use a standard syringe having a solid plunger. The solid plungers of automated instrumentation employing multiple syringes may be replaced with the inventive plunger so that the syringes can be easily cleaned and reagents can be added reducing the washing times and reducing or eliminating cross contamination.

### CROSS-REFERENCE TO RELATED APPLICATIONS

This application claims the benefit of U.S. Provisional Application 60/550,551 filed Mar. 5, 2004.

### STATEMENT REGARDING FEDERALLY SPONSORED RESEARCH OR DEVELOPMENT

Not Applicable.

### BACKGROUND OF THE INVENTION

1. Field of the Invention

The present invention relates to syringes used in biotechnology, biochemistry or clinical research, and more specifically to those applications wherein there is a requirement for handling liquids or materials turned into liquid from semisolid or solid by a chemical or physical process, in small and large measured volumes for the purpose of aspirating and dispensing liquid samples.

2. Description of the Related Art

Mechanical devices, such as syringes, for aspirating and dispensing a predetermined volume of liquid are known in the art. Such devices are commonly used by industry, hospitals and research facilities throughout the world. The predetermined volume of fluid may vary from nanoliters to gallons, but most laboratory settings use volumes ranging from nanoliters to several milliliters.

Typically a measuring device, such as a pipet or syringe is inserted into a container for a sample or reagent. A volume of fluid is aspirated or drawn into a pipet or syringe, which is then withdrawn and moved to a second container. The sample or reagent is dispensed from the syringe into the second container. Such aspiration and dispensing of fluid or reagent is accomplished through a needle or nose section of a syringe or pipet.

Single syringes, pipettors, or units containing multiple such devices, such as multi-channel pipettors are designed to transfer liquids from containers of one size to containers of another size. While a single syringe can be used to accomplish multiple such transfers,

A typical syringe includes a hollow barrel fitted with a solid plunger. One or both ends of the barrel are fitted with an end fitting, which can accept other analytical devices such as a valve, a needle, or a tube. The plunger fits within the barrel and includes a seal to maintain the liquid within the area defined by the barrel, the end fitting and the end of the plunger located within the barrel. The plunger usually has a fixed button-like knob at the end extending from the barrel, which button is used to force the plunger in and out of the barrel.

Prior art syringes and pipettors including plungers having an orifice therein:

U.S. Pat. No. 2,602,447 issued to Kollsman on Jul. 8, 1952. This patent discloses an injector ampule in which a slot is formed into the plunger. The slot is only deep enough that a corresponding plug on a seal member fits snugly within the slot. No fluid is communicated to the slot.

U.S. Pat. No. 4,089,335 issued to Harris on May 16, 1978 discloses a microsyringe comprising a barrel provided with an axial bore, at the forward or outlet end of which is affixed a hollow needle, and within the opposite end of which is fitted a slidable plunger. The forward terminal end of the plunger is hollow and provided with a fixed seal. A plug on the fixed seal fits within the hollow orifice of the plunger. No fluid is communicated to the hollow orifice of the plunger.

Neither of these prior art syringes have orifices through the entire plunger and in fluid communication with the syringe chamber.

When changing gas or liquid handling contents, the plunger in prior art syringes and pipettors are forced up and down to remove all unwanted contents or flushed by withdrawing some external fluid from the tip, which is usually immersed in a liquid. The up and down stroke removes liquid from the barrel cavity and also can be used to wash or cleanse the syringe from its prior contents. This typically takes several strokes to accomplish and is often unsuccessful. Such is also the case with manual and automated equipment or multichannel syringe configured instruments.

In a complex analytical procedure typical of many fields of chemistry, biology and biotechnology, the cleaning operation must be performed hundreds of times across replicate samples and across multiple aspirations-transfer-dispense sequences. The containers in which this process occurs are typically large geometric arrays of small volume vessels. Due to the number of samples or repetitions that must be performed, multiple channel pipettor units were designed to give higher throughput and reduce time required for the task. Therefore, inventors and manufacturers have created instruments for multiple channel or multiple probe liquid handling that are able to aspirate, transfer and dispense liquids from one to 96 channels, or more, at once. Attached to each individual channel or probe is a disposable tip, or needle. In the case of automated equipment, disposable tips or needles are often attached to each individual probe, although some equipment uses syringes with fixed or replaceable needles.

Over time, continued use of a plunger can cause wear on the inner surface of the syringe or pipettor to the extent that it cannot be replaced. It would be an improvement to the art to provide a plunger that could reduce the number inward and outward cycles necessary for performing non-productive tasks, such as syringe cleaning, that are required to ensure the integrity of the productive tasks.

Sampling contamination is prevalent and the analytical time to sample from one channel to another can be great. It would be an improvement to the art to provide a plunger that can reduce the amount of time sampling between channels.

Often, technicians must perform precise movements and very accurate placement of pipet tips or sample syringe needles hundreds of times per day, causing considerable stress over time leading to such work-related injuries as carpal tunnel hand syndrome. It would be an improvement to the art to provide a plunger that reduced the number of times inward and outward plunger movements were required to perform the required work each day.

Another area in which prior art syringes and pipettors may be improved relate to cleaning the inner orifice of the syringe or pipettor. Syringes are designed to pull up or aspirate a sample, liquid, or gas. Upon removing the syringe contents, it is acceptable protocol to flush the syringe barrel with new liquid or gas to cleanse the barrel before the next sampling or reagent addition operation. This takes considerable time and is technically dependent on the ability of the technician. In the case of manual syringes, mistakes can be made and syringes can be broken due to excessive use even to the extent that they may need to be replaced. Automated equipment which is designed for higher throughput of samples need special flushing liquids often requiring several strokes of the plunger. Thus, more use of the syringe tip is expended resulting in lengthening the time of the analyses, which competes with the process of automation time. All of these objections are taken as everyday practices using the typical syringe which is designed and sold from many manufacturers and have been available for over 40 years.

### BRIEF SUMMARY OF THE INVENTION

Accordingly, it is an object of the present invention to provide a syringe plunger having an orifice therethrough that:
allows the addition of liquids or gases to the syringe through an upper end of the plunger for the purpose of cleaning syringe contents;
allows the addition of liquids to the syringe through an upper end of the plunger for the purpose of adding samples from an external vessel or tube;
allows the addition of liquids to the syringe through an upper end of the plunger for the purpose of adding reagents to syringe contents;
allows the addition of liquid or gas samples from a multi-port valve device through a top end of the plunger for the purpose of adding multiple components to the syringe and cleaning syringe contents;
allows the addition of liquids through the top end of a mechanical plate used in automated and semi-automated instruments for the purpose of cleaning or adding reagents;
attaches or is retrofitted onto hand-held liquid/gas delivery devices, automated equipment having syringes, or semi-automated pipettors;
is controlled by a computer or switch-controlled mechanism incorporated on automated equipment;
allows the addition of liquids or gases from the needle end of the syringe up through the top of the hollow plunger using an auxiliary valve at the needle end by closing it off after drawing in the liquid or gas and opening the upper valve when the plunger is pushed forward;
allows probes, needles or disposable tips to be oscillated within liquid vessels or micro plates at predetermined depths without requiring excessive plunger movement;
attaches quickly and simply to preexisting equipment without requiring specific integrated assemblies or systems to adapt the syringe plunger to the equipment;
enhances functionality of existing equipment; and
reduces or eliminates the strain on hands, wrists, arms, and shoulders while performing liquid or gas handling tasks handling tasks.

Other features and advantages of the invention will be apparent from the following description, the accompanying drawing and the appended claims.

This invention is for a plunger, for use with a syringe-like device, having an orifice therethrough providing fluid communication along the length of the plunger. A valve may be located at one or both ends of the plunger to manipulate fluid flow through the plunger and into the syringe. A valve may be located at the needle end of the syringe to manipulate fluid flow or gas control alone or in addition to one or more valves on the plunger. The size of the orifice may vary depending upon the nature of the fluids being transmitted through the plunger and the syringe size. Plungers for multiple syringe devices, pipets, and pipettors have orifices therethrough. A plurality of tubing interfaces with the plurality of plungers to provide fluid communication from one or multiple sources to the syringes, pipets, or pipettors.

In accordance with the present invention, a mechanical accessory in the form of a device which is an integral part of a syringe-type instrument is disclosed. A plunger, which has a hollow tube with an orifice capable of delivery of gases or liquids, in the form of a reagent, a sample, or cleaning agent may be component of a handheld syringe device, or a handheld liquid handling instrument or pipettor or automated instrumentation. Multi-port accessory devices may be added to enhance the addition of liquids or samples through the hollow plunger fitted with additional sampling valves, This invention is for syringes requiring the tasks of common daily aspiration and dispensing of liquids or gases that can be performed quicker and with more accuracy and ease.

### BRIEF DESCRIPTION OF THE DRAWINGS

FIG. 1 is a cross sectional view of a syringe with the inventive plunger.

FIG. 2 is a cross sectional view of a syringe body and inventive plunger.

FIG. 3 is a cross sectional view of a syringe body and inventive plunger with external tubing and an external plunger valve.

### DESCRIPTION OF THE INVENTION

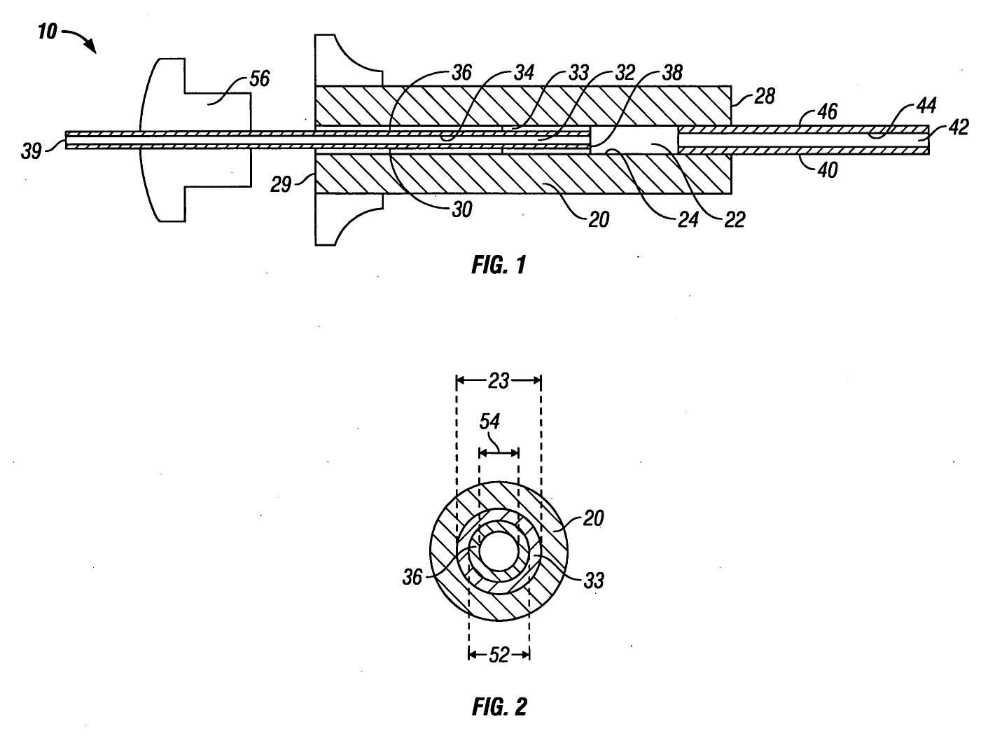

Referring to FIG. 1, the inventive syringe is depicted as 10. The syringe 10 comprises a syringe body 20, a plunger 30, and a needle 40.

The syringe body 20 is cylindrical in shape having an orifice 22 therethrough which defines an inner wall 24. Syringe body 20 may be graduated, but it is not necessary that it be so.

Extending from a first end 28 of syringe body 20 and fixed therein is needle 40. Needle 40 also has an orifice 42 therethrough. Needle 40 is cylindrically shaped, having a needle inner wall 44 and a needle outer wall 46. Needle outer wall 46 fits within inner wall 24. Needle 40 is secured within syringe body 20 such that there is no fluid communication between inner wall 24 and needle outer wall 46. A sealant may be used to ensure that there is no such fluid communication.

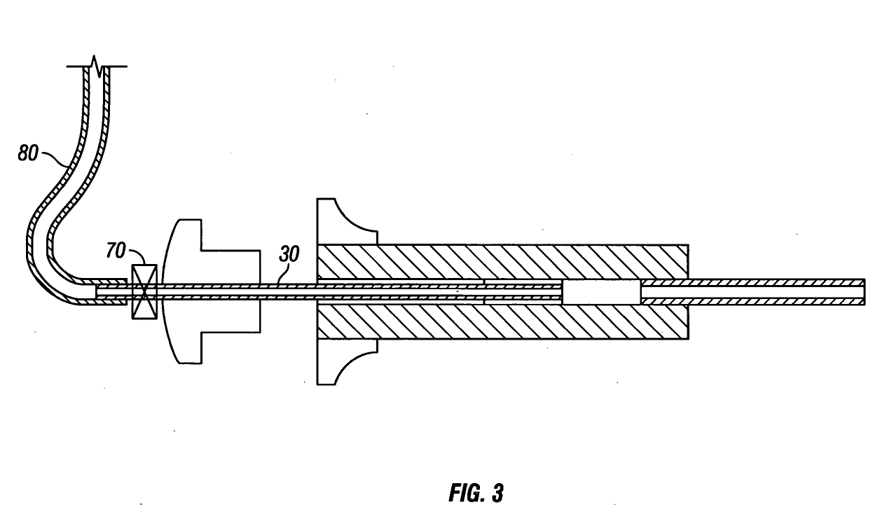

Referring to FIGS. 1 and 2, plunger 30 is cylindrically shaped and slidably received by a second end 29 of syringe body 20. Plunger 30 has an orifice 32 therethrough providing fluid communication between a first plunger end 38, which is fitted within a first orifice opening 26 in second end 29 of syringe body 20, and a second plunger end 39, which is external syringe body 20. Plunger orifice 32 defines a plunger inner wall 34. Plunger outer wall 36 has a diameter 52, which is smaller than inner diameter 23 of syringe inner wall 24.

A seal 33 is affixed to first plunger end 38 to prevent fluid communication between inner wall 24 of syringe body 20 and plunger outer wall 36. Seal 33 forms an interference fit between syringe inner wall 24 and plunger outer wall 36, although seal 33 is slidable along syringe inner wall 24. Plunger 30 is slidable within syringe body 20 between a fully compressed position, in which first plunger end 38 is against needle 40, and a fully extended position in which first plunger end 38 is in syringe body 20 only so far as is necessary to remain there. Plunger 30 may be removable from syringe body 20, however, it is not necessary that it be so.

Referring to FIG. 3, commercially available external tubing 80 may be attached to plunger second end 39. External tubing 80 may provide fluid communication between an external vessel (not shown) and plunger 30. Thus, plunger 30 may be effectively lengthened and connected to other analytical device attachments (not shown).

An external plunger valve 70 may be located along a portion of plunger 30 that remains external to syringe body 20 even when plunger 30 or an extension of plunger 30 (not shown) is fully compressed within syringe body 20. Valve 70 may be adjustable such that fluid communication between external tube 80 and plunger 30 is regulated and managed at a predetermined flow rate, cycled between a faster and a slower flow rate, or responsively changes the flow rate based on pressure changes at orifice 32 at first plunger end 38 or other external stimuli. It is understood by those skilled in the art that the term fluid, as used herein, refers to both liquids and gases.

Rather than changing the flow rate, external plunger valve 70 may stop or start fluid communication within plunger 30. When fluid communication is stopped, outward and inward movement of plunger 30 within syringe 20 will cause fluid to be drawn into or expelled through needle 40 due to pressure variation within syringe orifice 22. This action is similar to prior art syringes, however, some fluid may have been within plunger 30 when valve 70 was closed. Without pressure to maintain the fluid in plunger 30, it may flow into syringe orifice 22 and/or needle 40. The viscosity of the fluid in plunger 30 and the plunger orifice diameter 54 will also determine whether and the extent to which fluid from plunger orifice 32 will be transmitted to syringe orifice 22 or needle orifice 42.

When external plunger valve 70 is opened, fluid may flow within plunger 30. Outward and inward movement of plunger 30 within syringe 20 will cause pressure variation in syringe orifice 22. When plunger 30 is moved outward, the pressure within syringe orifice 22 drops and fluid is drawn into the area of lower pressure. The fluid may be drawn through needle orifice 42 and/or plunger orifice 32. Several variables will determine from where and to what extent fluid will be drawn into syringe orifice 22 when plunger 30 is moved outward toward the extended position. Among these variables are the relative orifice diameters of needle orifice 42 and plunger orifice 32, the relative viscosities of fluids being drawn through needle 40 and plunger 30, and the relative lengths of plunger 30 and needle 40. In addition, if there is pressure being applied to the fluid into plunger 30 or needle 40, that will be a factor as well. The additional pressurization from the external vessel on the fluid being transmitted to plunger 30 will be a factor in fluid flow into syringe orifice 22 upon extraction of plunger 30 from syringe 20.

When plunger 30 is moved inward, the pressure within syringe orifice 22 increases and fluid is expelled from the syringe orifice 22, assuming there is a passageway open for such expulsion. With external plunger valve 70 in an open position, such a passageway exists. Fluid may be expelled through plunger orifice 32 and/or needle orifice 42. As previously described, several variables will determine whether fluid is expelled through needle 40, plunger 30 or both. These variables include the relative diameters of plunger orifice 32 and needle orifice 42 as well as the relative lengths of each. Pressurization on the plunger orifice 32 and/or needle orifice 42 is also a factor.

If a valve that varies fluid flow is used as external plunger valve 75, then the extent to which the fluid flow is varied will also be a factor in the amount of fluid drawn into syringe orifice 22 or expelled therefrom.

An internal plunger valve 75 may be located at first plunger end 38 inside syringe 20. Internal plunger valve 75, like external plunger valve 70 may be of the type that adjusts fluid flow or stops and starts fluid flow either manually or based upon other stimuli such as pressure changes, flow rate changes, or even temperature changes.

The function of internal plunger valve 75 is similar to that described for external plunger valve 70.

A nose valve 60 may be located at or near the interface between needle 40 and first end 28 of syringe body 20. Nose valve 60 may be physically located at the interface between needle 40 and syringe body 20 or along needle 40 alone. Nose valve 60 may be opened to allow fluid flow through needle 40 or closed to prevent such fluid 1 communication.

External plunger valve 70, internal plunger valve 75 and nose valve 60 may each be incorporated into the overall syringe design individually or in combination. A combination of valves, such as an external plunger valve 70 and a nose valve 60 may be incorporated to have desired control of fluids being drawn into and expelled from syringe orifice 22. For example, nose valve 60 may be closed and external plunger valve 70 while plunger 30 is moved outward from syringe 20. This combination would allow fluid to flow through plunger orifice 32 into syringe orifice 22. All three valves, external plunger valve 70, internal plunger valve 75 and nose valve 60 may be incorporated into a syringe permitting additional control of fluid source and flow into and out of syringe orifice 22.

In an alternative embodiment, multiple external tubes 80 may be placed in selective fluid communication with plunger orifice 32. External tubes 80 may be connected to external vessels (not shown) containing different fluids. A valve, such as a rotary valve, may be used to select one or more fluids to be transmitted through plunger orifice 32 and into syringe orifice 22.

In an alternative embodiment, a plurality of plungers 30 may be used in connection with multiple channel measuring instruments, such as pipetors or multi-channel pipettors (not shown). External tubing 80 provides fluid communication from one or more external vessels (not shown) to plungers 30. One or more valves may be used, as previously described, to provide control of fluid communication within plunger orifice 32.

The use of the inventive plunger 30 is dependent upon the application and has many multiple uses. Following are two classic examples of how plunger 70 may be used.

Single Use

A syringe 20 has a small needle 40 located at first end 28 and plunger 30 slidable through second end 29. Plunger 30 is fitted with an external valve 70. A small amount of fluid is withdrawn from a given vessel (not shown). A rack (not shown) is filled with multiple samples so cross contamination is possible, which is a critical issue. The contents of syringe 20 are expelled. With the valve open and using an external pump, or pressured source, liquid or gas is forced through plunger orifice 32 into syringe orifice 22 when the plunger 30 pulled back to the maximum limit. This technique flushes or cleans the entire barrel length of syringe orifice 22. The external plunger valve 70 is closed and the fluid in the syringe orifice 22 is exhausted into a waste container. This process is repeated after each sampling.

To sample through the extended plunger 30 from a vessel (not shown) that is in fluid communication with syringe orifice 22 located at syringe first end 28 (or needle orifice 42), sample loading is performed by closing the external plunger valve 70 and aspirating the liquid from the vessel. The nose valve 80 is closed and the external plunger valve 70 is opened. The extended plunger 30 may then be pushed forward or inward. This action forces the fluid through plunger orifice 32 and out to another vessel (not shown). The syringe orifice 22 may then be flushed with new liquid by repeating the process as if the wash liquid was a sample.

Multi-Use

Multi-well and multiple tube liquid handling can be done using an array of several syringes 20 lined up with a special spacing for the appropriate application. The plungers 30 usually have a fixture or a “button” 56 (shown on FIG. 1) near plunger second end 39 so a top set of plates can capture the assembly and move the plungers 30 up and down with the plate assembly. The flushing of the syringes 20 can be accomplished with a multi-port manifold or valve with an external fluid delivery source. The same operation takes place when the external plunger valve 70 is opened or a pump (not shown) is started and the syringes 20 are cleaned or in some cases, reagents are added.

The foregoing description of the invention illustrates a preferred embodiment thereof. Various changes may be made in the details of the illustrated construction within the scope of the appended claims without departing from the true spirit of the invention. The present invention should only be limited by the claims and their equivalents.

1. A device used as an liquid or gas sampling device comprising of:
(a) A hollow plunger fitted to a glass or polymeric barrel, and
(b) the orifice in the ends of the device being sized to accommodate the needed reagent, and,
(c) the device connected to a platform which will hold syringes in a spatial configuration or plates or tubes ready for liquid handling analytical tasks, and,
(d) the device made of metal and/or plastic components such that integration of said components have the ability to interface with a valve or on/off analytical component, and
(e) by means of movement of components along an axis, will be able to sample and add external fluids through the given orifice and,
(f) the device can be sized in dimension for different scale application requirements.
